(19) United States
(12) Patent Application Publication
(10) Pub. No.: US 2013/0056551 A1
(43) Pub. Date: Mar. 7, 2013

(54) RELEASABLY ENCAPSULATED AROMA

(71) Applicants: Naijie Zhang, Ridgefield, CT (US); Peter S. Given, JR., Ridgefield, CT (US)

(72) Inventors: Naijie Zhang, Ridgefield, CT (US); Peter S. Given, JR., Ridgefield, CT (US)

(73) Assignee: PepsiCo, Inc., Purchase, NY (US)

(21) Appl. No.: 13/223,834

(22) Filed: Sep. 1, 2011

(51) Int. Cl. A61L 9/04

(52) U.S. Cl. 239/60

(57) ABSTRACT

Gelatin capsules encapsulating an aroma material including at least one aroma compound may be applied to product packaging, such as food packaging etc., with a secondary protective coating, e.g., at the interface of a container and its closure device. The gelatin capsules can be ruptured or broken when the container is opened, thereby releasing the aroma compound and causing a favorable aroma for the consumer. The secondary protective coating can reduce or prevent degradation of the gelatin capsules during product packaging, transport, and storage, thereby enhancing their performance.

### FIELD OF THE INVENTION

The invention relates to an aroma delivery system. In particular, the invention relates to an aroma delivery system comprising one or more polar, non-polar and/or volatile aroma compounds encapsulated in gelatin capsules. Product packages and the like comprising such releasable aroma also are provided.

### BACKGROUND OF THE INVENTION

Consumers evaluate many products by the aroma emitted from the product or the container in which the product is made available. Some consumers prefer to perceive a characteristic product odor each time the package is opened. Edible products, such as juices and coffee, are expected to have a fresh aroma that replicates or evokes memory of the expected flavor of the product. Research has shown that aromas can in some instances have substantial impact on consumer perception of the taste of a beverage or other food, trigger a favorable emotional response, elicit a favorable memory, and/or otherwise improve overall product performance. Inedible consumer products such as personal care products also are evaluated by the aroma. For example, consumers seek mouthwashes that provide a ‘fresh’ aroma and deodorants, for example, that provide a selected effect, such as ‘fresh’ or ‘sport’. Laundry detergents and fabric softeners also may provide such an effect.

When a beverage or other food container is first opened, it is often desirable that the consumer perceives the aroma of the food. Because the food's ingredients usually determine its aroma, those ingredients can be selected to provide a pleasant aroma, as well as the desired taste characteristics. In some cases it may be problematic, however, to make use of this phenomenon without modifying the ingredients to include aromatic compounds that would adversely affect the taste of the food or beverage. Therefore, packagers have attempted to design containers that, for example, release an aromatic substance when the container is opened. Similar aroma release may be necessary or useful for other consumer products as well. For example, because the aroma of the product is a significant factor used by consumers when selecting personal care products, consumers commonly attempt to open personal care products to smell the fragrance of the product before deciding to purchase. The quality or impression created often leads to an immediate decision on whether to purchase a product.

The aroma of a product often is not adequately revealed when the consumer opens the container because the orifice through which a product is dispensed is small, or a safety film is used under the cap to protect the integrity of the product. Additionally, it often is difficult to deliver adequate aroma to a headspace of a container that comes from the beverage itself, and not from the container. Therefore, overwraps that release aroma and strips on the outside of the container that release aroma, also known as ‘scratch and sniff’ strips, have been used to deliver aroma to consumers. However, overwraps, once breached, may present an unsatisfactory appearance to the consumer and typically are not effective in retaining an aroma. Devices placed on the outside of packages are also not always satisfactory for a consumer who expects to perceive the aroma from the product itself when the container is opened, rather than from the outside of its container. Also, consumers often do not have confidence that ‘scratch-and-sniff’ devices accurately portray the aroma of the product. Therefore, consumers tend to open the cap or closure of a product's packaging, seeking to experience the product's aroma.

Thus, there exists a need for an aroma delivery system for consumer products of diverse types. In particular, there exists a need for an aroma delivery system for delivery of aroma materials that are polar, hydrophilic, or volatile.

### BRIEF SUMMARY OF THE INVENTION

A first aspect of the invention is directed to an aroma delivery system that comprises gelatin capsules each having a surface and formed of material comprising gelatin. An aroma material comprising an aroma compound (i.e., at least one compound effective to create an aroma when released into the air) is releasably encapsulated in the gelatin capsules. A secondary protective coating is provided on the surface of the gelatin capsules to protect the capsules and the aroma compounds encapsulated in them from degradation, e.g., due to exposure to moisture, oxygen, etc. The gelatin capsules can be disposed on product packaging, e.g., on a beverage bottle, other food container, etc., to be ruptured or broken open during opening of the package. The secondary protective coating can be provided on the surface of the gelatin capsules after applying the capsules to the packaging. The capsules are ruptured or broken open during opening of the package, typically, for example, by friction or impact contact between surfaces of the packaging, e.g., between the surface of a container and an adjacent surface of a closure device for the container, which closure device is moved relative to the container to open the container. In certain embodiment, for example, the gelatin capsules with secondary protective coating are applied at the interface of a bottle cap and the neck of the bottle such that pulling or twisting the cap off ruptures at least some of the gelatin capsules and releases aroma compounds to be favorably perceived by the consumer at that point of consumption.

In certain exemplary embodiments, the aroma material includes at least a compound selected from the group consisting of a volatile compound, a polar compound, a hydrophobic compound, and combinations of any of them. The aroma compounds typically are volatile, and encapsulating in the gelatin capsules applied to a product package can preserve them for release when the package is opened by the consumer. However, gelatin capsules are moisture sensitive and lose their performance associated aroma character change when the capsule is stored under high humidity conditions, e.g., the high relative humidity conditions often existing during transport or storage of food packages. In accordance with this disclosure, the performance of gelatin capsules containing aroma material, e.g., their stability, is improved by a secondary protective polymer coating on the surface of the gelatin capsules. The secondary protective coating of the aroma delivery systems disclosed here can reduce or eliminate degradation of the aroma material during use, storage, shipment, etc., e.g., when the gelatin capsules are on product packaging. In certain exemplary embodiments, the secondary protective coating comprises or consists essentially of a polysaccharide, a synthetic polymer, a natural wax, a natural biopolymer, a natural film former, other suitable material of or a combination of any of them.

In another aspect, a container is provided having an openable closure, for example a hand-openable closure, e.g., a beverage bottle with a threaded or otherwise removable and re-closable cap, and an aroma delivery system as disclosed above. The delivery system is on a surface of the closure or is otherwise associated with the closure to release at least a portion of the encapsulated aroma when the closure is opened. In certain exemplary embodiments, a sufficient quantity of the delivery system is disposed at the interface of a beverage bottle and its threaded or otherwise removable and re-closable cap, that a portion of the delivery system is released each of multiple times (e.g., at least the first two times) the cap is removed from the bottle.

These and other aspects, advantages and features of the technology disclosed here will become further apparent from the following more detailed description of certain exemplary embodiments or versions of the delivery systems and product packaging It should be understood that the various alternative embodiments or versions and optional features of the various embodiments described here, and others that are apparent from such description are not mutually exclusive. Rather, all embodiments having any or all compatible combination or permutation of those features are contemplated as within the scope of this invention.

### BRIEF DESCRIPTION OF THE DRAWINGS

FIG. 1 shows embodiments of the invention on a threaded closure.

FIG. 2 shows embodiments of the invention on a snap closure.

FIGS. 3A and 3B are micrographs demonstrating improved stability for gelatin capsules having a secondary coating in accordance with one embodiment of the invention disclosed here.

### DETAILED DESCRIPTION OF THE INVENTION

Various examples and embodiments of the inventive subject matter disclosed here are possible and will be apparent to the person of ordinary skill in the art, given the benefit of this disclosure. In this disclosure reference to “certain exemplary embodiments” (and similar phrases) means that those embodiments are merely non-limiting examples of the inventive subject matter and that there likely are other alternative embodiments which are not excluded. Unless otherwise indicated or unless otherwise clear from the context in which it is described, alternative elements or features in the embodiments and examples below and in the Summary above are interchangeable with each other. That is, an element described in one example may be interchanged or substituted for one or more corresponding elements described in another example. Similarly, optional or non-essential features disclosed in connection with a particular embodiment or example should be understood to be disclosed for use in any other embodiment of the disclosed subject matter. More generally, the elements of the examples should be understood to be disclosed generally for use with other aspects and examples of the devices and methods disclosed herein. A reference to a component or ingredient being operative, i.e., able to perform one or more functions, tasks and/or operations or the like, is intended to mean that it can perform the expressly recited function(s), task(s) and/or operation(s) in at least certain embodiments, and may well be operative to perform also one or more other functions, tasks and/or operations. While this disclosure includes specific examples, including presently preferred modes or embodiments, those skilled in the art will appreciate that there are numerous variations and modifications within the spirit and scope of the invention as set forth in the appended claims. Each word and phrase used in the claims is intended to include all its dictionary meanings consistent with its usage in this disclosure and/or with its technical and industry usage in any relevant technology area. Indefinite articles, such as “a,” and “an” and the definite article “the” and other such words and phrases are used in the claims in the usual and traditional way in patents, to mean “at least one” or “one or more.” The word “comprising” is used in the claims to have its traditional, open-ended meaning, that is, to mean that the product or process defined by the claim may optionally also have additional features, elements, etc. beyond those expressly recited.

In certain exemplary embodiments, an aroma delivery system comprises an aroma compound releasably encapsulated in gelatin capsules. The gelatin capsules have a coating, referred to here as a secondary protective coating.

In certain exemplary embodiments, the aroma delivery system is associated with product packaging in a manner that it at least a portion of the encapsulated aroma is released when the package is opened. For example, the delivery system can be associated with a cap, plug, seal or other type of closure of a package. In certain exemplary embodiments the closure is openable and re-closable. In certain exemplary embodiments the delivery system is associated with the product packaging by being carried on a surface of the container and/or of the closure device, e.g., at the interface between them, in a manner that causes release of the encapsulated aroma material (and, so, of the aroma compound(s) included in the aroma material), thereby releasing the desired aroma to be perceived by the consumer. Optionally a sufficient quantity of the delivery system is provided such that aroma is released each time the container is opened, multiple times, e.g., two or more times, three or more times, etc. In certain exemplary embodiments of the invention, the aroma is released when the gelatin capsule and the secondary protective coating are breached or broken and the aroma compound in the gelatin capsule is exposed to the atmosphere. Thus, embodiments of the invention can be applied to a screw-top container, to a flip-top container, or to any friction-type closure that, upon opening, will breach or break at least some of the gelatin capsules and the secondary protective coating to expose the aroma compound, and allow the release of the aroma. The delivery system can be applied, e.g., to the threads of a screw-on cap, to the contact area of a snap-on being inserted or removed.

Also, multiple different embodiments of the delivery systems disclosed here can be applied to a product's packaging, whereby a different aroma is achieved upon one opening of the package and a different aroma is achieved upon a subsequent opening. In such embodiments, for example, the different delivery systems can be layered one on another, spatially separated, or other associated with the packaging.

Certain exemplary embodiments of the beverage container products include ready-to-drink beverages, beverage concentrates, syrups, shelf-stable beverages, refrigerated beverages, frozen beverages, and the like; carbonated and non-carbonated soft drinks, liquid concentrates, fruit juice and fruit juice-flavored drinks, sports drinks, energy drinks, fortified/enhanced water drinks, soy drinks, vegetable drinks, grain-based drinks (e.g., malt beverages), fermented drinks (e.g., yogurt and kefir), coffee beverages, tea beverages, dairy beverages, and mixtures thereof. Beverage container products include bottles, cans, and carton products and fountain syrup applications. Embodiments of the invention can be useful for food containers for foods other than beverages including, e.g., snacks, cakes, cookies, baked goods, fermented food products, yogurt, sour cream, cheese, salsa, ranch dip, fruit sauces, fruit jellies, fruit jams, and fruit preserves.

In certain exemplary embodiments, an aroma compound is entrapped in a gelatin capsule. The aroma compound typically is selected to provide the aromatic experience expected by the user by providing an aroma that is representative of, and congruent with, the product in the container. The gelatin capsule is selected to entrap the aroma compound, protect the aroma compound from degradation and premature or unintended release, and release the aroma when the gelatin capsule is breached or broken.

The precise aroma used with a product will vary, depending on the intended effect. For example, products that desireably have a ‘fresh’ or ‘sport’ aroma, likely will have that type of aroma encapsulated for delivery from an aroma delivery system embodiment of the invention. Similarly, edible products typically will have an aroma delivery system that enhances or compliments the natural aroma of the edible product, such as a coffee aroma, a fresh fruit aroma, or a beverage flavor aroma.

Polar and non-polar aroma compounds can be encapsulated in the aroma delivery system of the invention. Certain exemplary embodiments of the invention are directed to an aroma delivery system in which the aroma compound is polar, hydrophilic, non-polar, hydrophobic, or volatile. The aroma material in certain embodiments is a solid or a powder. The skilled practitioner will recognize from this disclosure that an aroma delivery system capable of delivering polar, hydrophilic, and more volatile compounds may in at least certain embodiments also be capable of delivering a non-polar or hydrophobic aroma compound. Other embodiments of the invention are directed to the release of an aroma from volatile or polar (or hydrophilic) aroma compounds. Still other embodiments of the invention are directed to aroma delivery systems applied to containers used for food and beverage packaging applications. Certain exemplary embodiments are directed to the entrapment of volatile polar aroma compounds.

In certain exemplary embodiments, the aroma compound (or at least one of them if multiple aroma compounds is included in the encapsulated aroma material) is polar or non-polar, hydrophobic or hydrophilic, and/or has other properties. In certain exemplary embodiments, the aroma compounds comprise perfumes of any type, including natural perfumes such as, for example, frankincense, and manufactured perfumes; essential oils, such as, for example, valencia, lemon, lime, grapefruit, tangerine, orange, and sandalwood; components of essential oils, such as, for example, limonene, citral, furaneol, vanillin, and other terpenes, sesquiterpenes, diterpenes, and oxygenated forms of these terpene compounds; and other fruit essences or aromas, such as, for example, cherry, pineapple, apple, and mango; or combinations of any of them. In certain embodiments of the invention, the aroma compound comprises a coffee aroma, comprising any one or more of the many aliphatic, acyclic, aromatic benzenoids, heterocyclics, and other compound types known to be present in coffee or coffee aroma. In certain embodiments, the aroma compounds may be used in combination.

Without wishing to be bound by theory, it is currently understood that the gelatin capsules with the secondary protective coating protect the aroma material and its aroma compound(s) from degradation and premature release. The gelatin capsules will then release the aroma in response to being breached or broken open, most typically by the action of opening the container. In certain exemplary embodiments of the invention, this breach or breaking is occasioned by contact between the product container and a surface of the closure device of the container, e.g., by friction during sliding contact between their surfaces or impact contact, etc.

In certain exemplary embodiments, the diameter of the gelatin capsules is in the range of 10 microns to 50 microns. It will be understood by those skilled in the art given the benefit of this disclosure, that suitable sizing for the capsules will be determined in part by the nature of the packaging and the mariner in which the capsules are associated with the packaging.

In certain exemplary embodiments, the gelatin capsules are selected for a particular usage to resist environmental conditions or circumstances that would degrade the gelatin capsules or the aroma compounds. For example, gelatin capsules used in food packaging typically should be selected to be resistant to moisture, food acids, and other ingredients in the container. Similarly, an aroma delivery system used in a container for a product that emits a vapor, such as a solvent, typically should be adequately resistant to that vapor. The gelatin capsule also may be chosen for compatibility with the physical and chemical properties of the aroma material, including the one or more aroma compounds, any solvents, diluents, carriers and other materials of the aroma material. The gelatin capsule also may be selected to protect the aroma compound encapsulated therein against heat, moisture, light, especially ultraviolet light, and other deleterious conditions.

In certain exemplary embodiments, i.e., non-limiting examples or version of the delivery systems disclosed here, the gelatin capsules encapsulating the aroma material are gelatin-hydrocolloid capsules. They can be formed, e.g., of a coacervate material comprising one or more gelatin and one or more anionic compounds, optionally with a cross-linking agent and/or other auxiliary materials. In certain embodiments the at least one anionic polymer may be selected from gum arabic, modified starch, pectin, alginate, and combinations of any of them. The complex coacervates of the gelatin capsules can be formed in accordance with known techniques. For example, they can be formed by combining a solution of the gelatin with the aroma material to form an oil-in-water emulsion, and then adding anionic polymer(s) to form the complex coacervate capsules, i.e., the gelatin capsules, encapsulating the aroma compounds. The gelatin capsules may be, for example, gelatin-pectin, gelatin-alginate, gelatin-gum arabic, gelatin-carboxymethyl cellulose, etc. Combinations of any one or more types of gelatin capsules may be used.

As disclosed above, the aroma delivery systems further comprise a secondary protective coating on the surface of the gelatin capsules. Such secondary protective coating is especially advantageous, for example, in certain embodiments with an aroma compound that is particularly volatile or sensitive to degradation, or has a tendency to becoming unstable during storage. In such embodiments, it is believed that the secondary protective coating inhibits or prevents aroma material leaking from the capsules and/or moisture or oxygen penetration into the capsules. In this way, the secondary coating on the capsules helps to prevent or reduce oxidation and, to maintain aroma character. The secondary protective coating helps to retain the integrity of the aroma compound and the aroma delivery system. In certain embodiments, the secondary protective coating provides additional resistance to moisture, and so is moisture resistant. Thus, the secondary protective coating may serve different purposes, and the composition of the coating thus can be selected to serve particular intended purposes.

In certain exemplary embodiments of the invention, secondary protective coatings comprise bio-polymers, polysaccharides such as pectin, agar, carrageenan, alginate, guar gum, xanthan gum, gellan gum, acacia gum, locust bean gum, gum ghatti, starch, modified starch, cellulose, and carboxymethylcellulose; synthetic polymers, such as polyvinyl alcohol, polyvinyl acetate, polyacrylates, polystyrene-acrylate, polysters, polyurethanes, polyureas, melamine/formaldehyde resins, and polysiloxanes; natural waxes, beeswax such as carnauba wax, candellila wax, shellac, and natural film formers such as natural shellac and corn zein protein; or combinations of any of them.

In certain embodiments, the secondary protective coating may be applied to a portion of the surface of the gelatin capsules. In alternative embodiments, the secondary protective coating may be applied to the entire surface of the gelatin capsules. In certain embodiments, the secondary protective coating may be applied to the gelatin capsules before they have been applied to the container, or alternatively, the secondary protective coating may be applied to the gelatin capsules after they have been applied to the container. In certain embodiments, the secondary protective coating may additionally cover a portion of the container, for example, to ensure that the edges of the gelatin capsules are sealed against degradation. The secondary protective coating may be applied to the gelatin capsules by spray coating, painting on, etc. In certain embodiments, the aroma delivery system comprising the secondary protective coating may be applied to a container for an edible product. In such embodiments, the secondary protective coating typically should comprise food-safe materials.

The delivery system is formed by encapsulating aroma material, e.g., aroma material comprising or consisting essentially of one or more aroma compounds, in the gelatin capsules and applying the secondary coating. Delivery systems in accordance with certain embodiments of the invention are made by encapsulating the aroma material in the gelatin capsules, for example, by mixing the aroma material with gelatin and an anionic polymer. The resulting capsules can be applied to a container, e.g., to the main body of the container and/or to its cap or other closure device. After drying, the applied, optionally with or without other materials, e.g., a binder or carrier, such as water or other liquid, a film forming material, a paste, adhesive, etc. In certain embodiments the aroma delivery system is applied as a liquid, powder, paste or other suitable form. In certain embodiments the aroma delivery system dries after being applied, to form a film on the surface(s) of the container. In certain embodiments the aroma delivery system is disposed on flat surface areas, in grooves, threads, nooks or crevices or the like, or a combination of any of those, on one or more surfaces of the container, optionally including one or more closure surfaces. Suitable binders (alternatively referred to here as a carrier) for applying the gelatin capsule to a product package prior to applying the protective secondary coating will depend, at least in part, on the particular application, including, e.g., the packaging material, its shape and/or intended usage. Exemplary binders suitable for various applications include vinylic or vinyl-containing polymers, e.g., polyvinyl alcohol, polyvinyl acetate, etc., and natural film forming polymers or bio-polymers, e.g., starch or other polysaccharides. In, some embodiments, edible or food safety approved materials may be used. Other suitable binders will be apparent to those skilled in the art given the benefit of this disclosure

In certain exemplary embodiments, an aroma delivery system as disclosed here is applied to the closure of a container and allowed to dry to form a film. The secondary coating is added after the gelatin capsules are on the closure and dried. In such embodiments the film is located on the closure so that removing the closure will abrade, breach, or break at least some of the gelatin capsules of the aroma delivery system and allow the aroma compound to escape when the container is opened. In certain embodiment the aroma-containing gelatin capsules are provided as an emulsion, such as an oil-in-water emulsion or slurry. Typically such oil-in-water emulsion embodiments can be applied onto the package by coating, spraying, printing, brushing, etc. Such methods of application will be suitable for other embodiments as well. Other suitable methods of application will be apparent to those skilled in the art given the benefit of this disclosure.

In alternative embodiments, a powder or solid aroma compound, such as furaneol or vanillin, may be melted by heating prior to adding and encapsulated into the gelatin capsules. In certain exemplary embodiments, the aroma material consists essentially of only one or more volatile aroma compounds. In other embodiments the aroma material may include aroma compounds in a solvent, carrier, diluent or the like. In such embodiments the combined, i.e., total concentration of the one or more aroma compounds in the aroma material encapsulated in the gelatin capsules may be, e.g., from about 1 to about 50 percent w/w, e.g., from 1 to 40 percent w/w, typically between about 10 and about 40 percent w/w, and more typically between about 20 and about 30 percent w/w.

In certain exemplary embodiments, the surface of the container may be treated or prepared to ensure that the aroma delivery system adheres sufficiently to the container. For example, it may be necessary in some applications to roughen the surface of a container formed from polyethylene terephthalate, or to apply an appropriate surface primer. Suitable primers for at least some embodiments can be selected from polyvinyl alcohol and polyvinyl acetate. Any suitable method of surface preparation may be employed to ensure sufficient adhesion of the applied aroma delivery system.

In certain exemplary embodiments, the aroma delivery system is applied to the closure, such as the cap, of the container. The aroma delivery system may be applied to a portion of the closure that is on the outside of the container, but under or within the closure. This placement causes breaching, breaking, scraping, or abrading of the delivery system to expose the aroma compound to the atmosphere and liberate the aroma so that the aroma is perceived by the consumer. The placement also protects the aroma delivery system from contact with external entities and ensures that the film is protected and not damaged by, for example, contact with other containers. In alternative embodiments, the aroma delivery system is applied to the inside of a container that has a closure that is torn open or may be crushed or crumpled upon opening, thus breaching, breaking, scraping, or abrading of the aroma delivery system to expose the aroma compound to the atmosphere and liberate the aroma so that the aroma is perceived by the consumer.

In certain embodiments, the container comprises a threaded cap on a neck, such as on a bottle. In such embodiments, the aroma delivery system can be applied to the outside of the neck and/or the inside of the cap, typically in the area of the threads that hold the cap on. The aroma delivery system will in such embodiments be located under the cap, so that the cap protects the delivery system during storage and transport, yet abrades, scrapes, breaches, or breaks the capsules when the cap is removed, thus releasing aroma.

In alternative embodiments, the aroma delivery system may be applied to a container having a closure, including a threaded closure having the threads on the inside of a neck of the container; a slide-in closure such as a cork or a stopper of metal, glass, or rubber, and including a stopper retained by a bail (a “Lightning” closure), or a “Baltimore loop;” or a snap-on or a slide-on cap or cover.

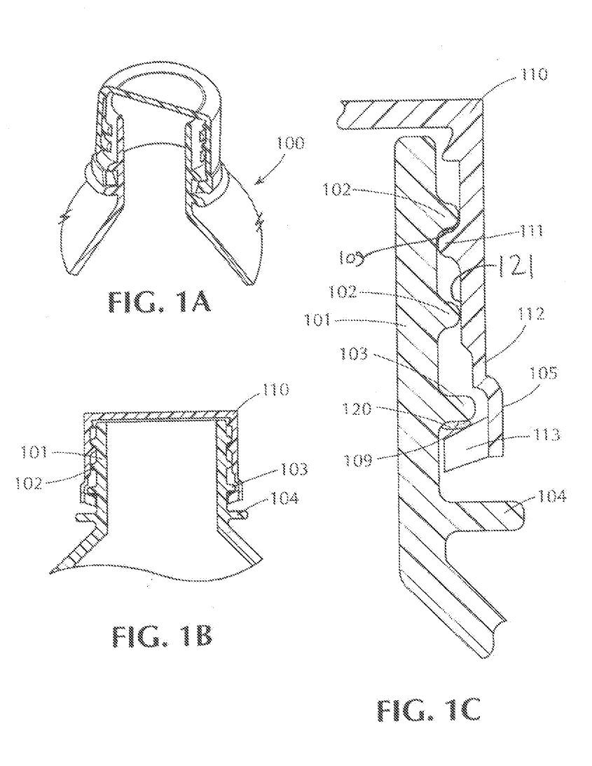

Drawing FIGS. 1A-1C illustrate an embodiment of the invention on a threaded cap. Container 100, such as a bottle, has neck 101 with spiral threads 102 formed on the outside thereof. Annular ring 103 also may be formed on neck 101 below threads 102. Flange 104 may be present to provide the used with support for pouring from the container.

Container 100 is closed by cap 110, which has interior threads 111 complementary to threads 102. Threads 111 cooperate with thread 102 to securely close container 100.

Cap 110 also may have a security feature 105 incorporating flange 113 that cooperates with ring 103. Security feature 105 may include a separation point at 112, so that cap 110 detaches from security feature 105 when the container is opened, or may otherwise indicate that container 100 has been opened, perhaps by deforming flange 113.

Delivery system 109 is applied to neck 101 in the area of threads 102 and 103 at a point where cap 110, and particularly threads 111, flange 113, or any combination thereof, when the cap is twisted off the container, rub against neck 101 and delivery system 109 thereon with force sufficient to release aroma from delivery system 109.

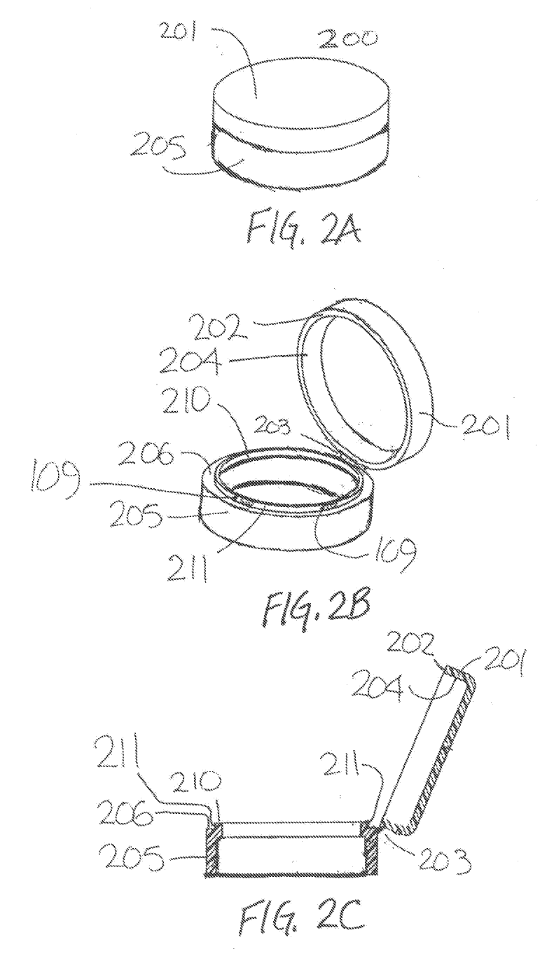

Drawing FIGS. 2A-2C illustrate embodiments of the invention for a snap-type closure having delivery system 109 applied thereto. Cap 200 has top 201 and body 205, and may be attached to each other by hinge 203. To close the container, top 201 is retained on body 205 by a friction or interference fit or snap fit with ridge 210. Top edge 202 typically contacts rim 206 and the top of ridge 210 contacts the interior of top 201 when the container is closed. Interior edge 204 of top 201 thus rubs outside edge 211 with force sufficient to break or breach delivery system 109 applied to outside edge 211 to release aroma therefrom. Delivery system 109 may be attached to all of or to any portion of outside edge 211.

In the examples provided, the delivery system may also be applied to inside portion 121 at locations where cap 110 rubs on threads 102, or on interior edge 204.

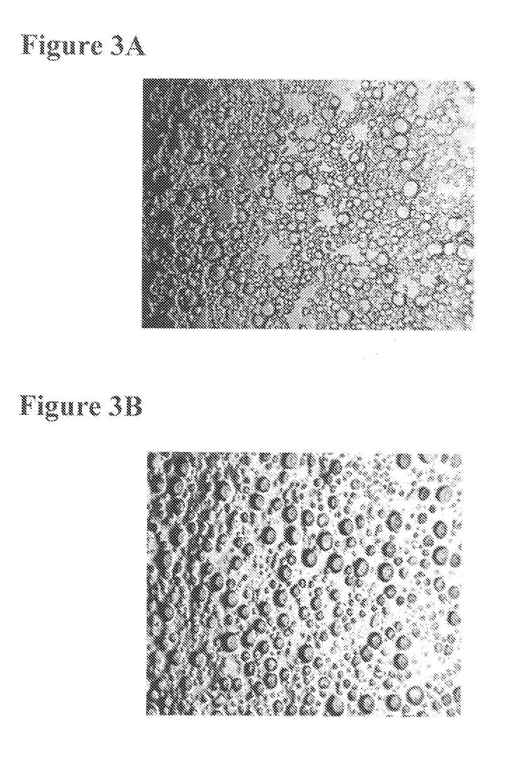

FIGS. 3A and 3B are micrographs showing improved stability for gelatin capsules having a secondary coating in accordance with an embodiment of this disclosure. FIG. 3A shows gelatin capsules containing liquid aroma material applied to a surface and then exposed to 92% relative humidity (RH) at room temperature (rt) for three days. Capsules are broken and areas of leaked liquid are evident, such that it can be seen that many of the capsules have ruptured, allowing the formerly encapsulated aroma material to escape. In contrast, FIG. 3B shows approximately the same density of the same gelatin capsules containing the same liquid aroma material (i.e., a second portion of the same batch of gelatin capsules) applied to the same surface (i.e., a different area of the same material) and then exposed to 92% relative humidity (RH) at room temperature (rt) for ten days. It can be seen that essentially all of the gelatin capsules are intact, i.e., they have not ruptured and they have not allowed the encapsulated aroma material to escape. Essentially no leaked liquid is present. Accordingly, the advantageous protection of the gelatin capsules and the encapsulated aroma material is demonstrated by the results this comparison shown in FIGS. 3A and 3B.

### Examples

The following Table 1 shows the results of testing of gelatin capsules containing aroma material in accordance with this disclosure, and comparative results for otherwise identical gelatin capsules having no secondary protective coating on the surface of the capsules. In each case the gelatin capsules were the same, being formed of gelatin complex coacervates with the encapsulated aroma material consisting essentially of lemon oil. The storage conditions in each case were the same, being 90 percent relative humidity at 25° C. The aroma intensity was judged by releasing the aroma material from the then-remaining gelatin capsules of the delivery system, by breaching or rupturing the capsules. It can be seen from the results in Table 1, that the secondary protective coating improved the performance of the aroma delivery systems. Those delivery systems that had a secondary protective coating in accordance with this disclosure are seen to have delivered greater aroma intensity than the delivery system without a secondary protective coating, even though the delivery system without a secondary protective coating was subjected to a much shorter storage duration of only 3 days instead of the two weeks storage of the invention embodiments.

TABLE 1
Gelatin Capsule Performance

| | Storage | | |
|---|---|---|---|
| Secondary Coating | Duration | | Aroma Intensity |
| None | 3 | days | very weak, not perceivable |
| Polyvinyl acetate | 2 | weeks | strong, perceivable |
| Shellac wax | 2 | weeks | strong, perceivable |
| Polyacrylate | 2 | weeks | strong, perceivable |
| Polystyrene-acrylate | 2 | weeks | strong, perceivable |
| Melamine- | 2 | weeks | strong, perceivable |
| formaldehyde resin | | | |
| Pectin | 2 | weeks | medium-strong, perceivable |
| Carboxymethylcellulose | 2 | weeks | medium-strong, perceivable |
| Polyvinyl alcohol | 2 | weeks | medium-strong, perceivable |
| Zein | 2 | weeks | medium-strong, perceivable |

It is believed that the aroma delivery system protects the aroma compound from oxidation or other degradation, prevents leakage, and maintains the aroma character. Various embodiments of the invention can provide a number of advantages over other aroma delivery systems, including easy handling and an easy manufacturing process, and good protection for the aroma material. The materials, i.e., the aroma compound(s), the gelatin capsule, and the secondary protective coating, used to construct the aroma delivery system optionally are food contact- or food-approved materials, as appropriate. Other advantages of at least some embodiment of the delivery system disclosed here include that the aroma delivery system is moisture resistant or even, in some cases, moisture-proof, colorless and/or transparent.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

We claim:

1. An aroma delivery system comprising:
gelatin capsules each having a surface and formed of material comprising gelatin;
an aroma material comprising an aroma compound that is releasably encapsulated in the gelatin capsules; and
a secondary protective coating on the surface of the gelatin capsules.

2. The aroma delivery system of claim 1, wherein the gelatin capsules comprise complex coacervates formed of gelatin and anionic hydrocolloid polymer.

3. The aroma delivery system of claim 1, wherein the gelatin capsules are formed of materials comprising gelatin-pectin complex coacervates, gelatin-alginate complex coacervates, gelatin-gum arabic complex coacervates or gelatin-carboxymethyl cellulose complex coacervates.

4. The aroma delivery system of claim 1, wherein the aroma compound is selected from a volatile compound, a polar compound, a hydrophobic compound, and combinations of any of them.

5. The aroma delivery system of claim 1, wherein the aroma compound is selected from an essential oil, a component of an essential oil, a fruit essence, a fruit aroma, a perfume, or combinations thereof.

6. The aroma delivery system of claim 1, wherein the secondary protective coating comprises a polysaccharide, a synthetic polymer, a natural wax, a natural biopolymer, a natural film former, or combinations thereof.

7. The aroma delivery system of claim 1, wherein the gelatin capsule has a diameter in the range of 10 microns to 50 microns.

8. The aroma delivery system of claim 1, wherein the combined concentration of the one or more aroma compounds in the aroma material encapsulated in the gelatin capsule is in the range of 1 to 50 percent w/w.

9. A product package comprising:
a container;
an openable closure for the container, and
an aroma delivery system associated with the closure to release aroma when the closure is removed from the container, the aroma delivery system comprising:
gelatin capsules each having a surface and formed of material comprising gelatin;
an aroma material comprising an aroma compound that is releasably encapsulated in the gelatin capsules; and
a secondary protective coating on the surface of the gelatin capsules.

10. The product package of claim 9, wherein the gelatin is selected from gelatin-pectin, gelatin-alginate, gelatin-gum arabic, gelatin-carboxymethyl cellulose or any gelatin-anionic hydrocolloid capsules.

11. The product package of claim 9, wherein the aroma compound is selected from the group consisting of a volatile compound, a polar compound, a hydrophobic compound, and combinations of any of them.

12. The aroma delivery system of claim 9, wherein the aroma compound is selected from an essential oil, a component of an essential oil, a fruit essence, a fruit aroma, a perfume, or combinations thereof.

13. The product package of claim 9, wherein the secondary protective coating consists essentially of one or more natural materials.

14. The product package of claim 13, wherein the secondary protective coating consists essentially of at least one natural biopolymer.

15. The product package of claim 9, wherein the secondary protective coating comprises a natural film forming material.

16. The product package of claim 9, wherein the secondary protective coating comprises polyvinyl alcohol, polyvinyl acetate, polyacrylate, polystyrene-acrylate, polyster, polyurethane, polyurea, melamine/formaldehyde resin, polysiloxane, biopolymer, polysaccharide; natural wax, bees wax, carnauba wax, candellila wax, shellac natural shellac, corn zein protein, or a combination of any of them.

17. The product package of claim 9, wherein the secondary protective coating comprises polysaccharide selected from pectin, agar, carrageenan, alginate, guar gum, xanthan gum, gellan gum, acacia gum, locust bean gum, gum ghatti, cellulose, carboxymethylcellulose and a combination of any of them.

18. The product package of claim 9, wherein at least a majority of the gelatin capsules each has a diameter in the range of 10 microns to 50 microns.

19. The product package of claim 9, wherein the concentration of the aroma compound in the gelatin capsule is in the range of 1 to 50 percent w/w.

20. The product package of claim 9, wherein the closure is a removable and re-closeable, hand-openable closure.

21. The product package of claim 9, wherein the closure has a surface having, an interface with a surface of the container and at least a portion of the aroma delivery system is positioned at the interface.

22. The product package of claim 9, wherein the closure comprises a threaded cap, a slide-in closure, a snap-on cap, or a slide-on cap.

23. The product package of claim 9, wherein the container is a bottle and wherein the closure is a threaded cap.

24. The product package of claim 23, wherein a portion of the aroma delivery system is released each of multiple times the threaded cap is removed from the bottle.
